(19) United States
(12) Patent Application Publication
(10) Pub. No.: US 2013/0079850 A1
(43) Pub. Date: Mar. 28, 2013

(54) EM Radiation eye protection systems and methods

(71) Applicant: Raphael Darvish, Los Angeles, CA (US)

(72) Inventor: Raphael Darvish, Los Angeles, CA (US)

(21) Appl. No.: 13/700,739

(22) PCT Filed: Jun. 20, 2011
(86) PCT No.: PCT/US2011/001095

§ 371 Date: Nov. 29, 2012

Related U.S. Application Data
(60) Provisional application No. 61/398,110, filed on Jun. 21, 2010.

(51) Int. Cl. A61N 5/06

(52) U.S. Cl. CPC A61N 5/0616; A61N 5/06; A61N 5/0617
USPC 607/89; 607/90

(57) ABSTRACT

Electromagnetic (EM) radiation eye protection systems (10) and methods are disclosed. The system includes an EM radiation source (20) and one or more pairs of protective eyewear (100). A control device (30) is operative to control the emission of an EM radiation beam (24B) from the EM radiation source in response to a transmit signal (WST1, ST1). Each pair of protective eyewear includes a sensor unit (120) that generates the transmit signal when the participant (P) properly wears the protective eyewear. The eye-protection system prevents the emission of the EM radiation beam if a participant is not wearing protective eyewear.

### CLAIM OF PRIORITY

This application claims priority from U.S. Provisional Patent Application Ser. No. 61/398,110, entitled “System to Ensure that Eye Protection is Being Worn by operator/participant/patient,” filed on Jun. 21, 2010.

### FIELD

The present disclosure relates generally to eye safety, and in particular relates to eye protection systems and methods for sources of electromagnetic (EM) radiation.

### BACKGROUND ART

EM radiation sources such as lasers and intense pulse light (IPL) sources are increasingly being used in a variety of applications that involve one or more participants. In the medical field for example, lasers and IPLs are used to treat a variety of medical conditions and to perform a variety of medical procedures. For example, lasers are used in dermatology for cosmetic purposes to remove wrinkles, tattoos, birthmarks, hair, to tighten skin, etc. The advantages of using lasers and IPLs to treat medical conditions and perform medical procedures are based mainly on the ability to direct controlled amounts of energy in the form of EM radiation to a specific location.

However, EM radiation poses a risk to the eyes of the participants, e.g., the EM radiation source user, the patient, and anyone else that happens in the treatment room or area, such as assistants, nurses, observers, etc. when performing the EM radiation-based procedure. Consequently, safety procedures need to be followed by the participants to mitigate the risk of eye damage.

One safety method is for the participants to wear protective eyewear while the EM radiation source is activated, i.e., is emitting an EM radiation beam. For this safety method to be effective, however, all of the participants need to remember to wear protective eyewear.

There is presently no system or method available that reliably ensures that each of the one or more participants in a radiation-based procedure is wearing protective eyewear. Thus, if any of the participants forgets to wear protective eyewear, there is a substantial risk of eye damage. Some EM radiation wavelengths are invisible to the naked eye so that a participant may not even know that eye damage is occurring since such an injury is generally painless. Eye damage in the form of retinal burns for example can be serious and can lead to a significant and permanent visual disability.

Further complicating matters is the fact that present-day protective eyewear used for EM radiation safety is comfortable to wear and has minimal if any practical visual impact. This has the unintended consequence of the participants losing situational awareness and not realizing whether they are or are not actually wearing the required protective eyewear.

### SUMMARY

The system and methods disclosed herein are directed to ensure that one or more participants involved in an EM radiation-based procedure, such as an EM radiation medical procedure, are wearing suitable protective eyewear while the EM radiation source is activated. This added layer of protection is needed because the various participants in an EM radiation-based procedure can easily forget to wear their protective eyewear during the procedure. This in turn can lead to eye damage for any of the unprotected participants.

An aspect of the disclosure is an eye protection system for a participant in an EM radiation-based procedure that uses an EM radiation source capable of emitting an EM radiation beam. The system includes a control device configured to be operably arranged relative to the EM radiation source. The control device is operable to control the emission of the EM radiation beam in response to a transmit signal. The system also includes protective eyewear having a sensor unit that is operable to transmit the transmit signal only when the protective eyewear is properly worn by the participant. The control device may be configured to matingly engage a remote interlock connector of the EM radiation source.

Another aspect of the system is the system as described briefly above, and that also includes the EM radiation source, with the control device operably arranged relative thereto.

In an aspect of the disclosure, the control device has first and second modules. The first module is configured to matingly engage the remote interlock connector, and the second module is configured to receive the transmit signal and relay it to the first module. The first module is configured to control the emission of EM radiation in response to receiving the control signal from the second module. This is accomplished, for example, by the first module having a switch that opens and closes in response to the control signal, which causes an electrical power circuit associated with the EM radiation source to be respectively disconnected and connected. In an example, the switch remains in the open position and closes in response to receiving the control signal.

Another aspect of the disclosure is an eye protection system for at least one participant in an EM radiation-based procedure that employs an EM radiation source. The system includes the EM radiation source, which is configured to emit an EM radiation beam suitable for use in the EM radiation-based procedure. The system also includes a control device operably arranged relative to the EM radiation source and configured to control the emission of the EM radiation beam. The system also includes at least one pair of protective eyewear for use by the at least one participant. The at least one pair of protective eyewear is configured to at least substantially block the EM radiation from the EM radiation source from reaching the at least one participant's eyes. The at least one pair of protective eyewear has a sensor unit and at least one activation element operably connected to the sensor unit. The at least one activation element is configured to cause the sensor unit to provide a transmit signal to the control device when the at least one protective eyewear is properly worn by the at least one participant. The control device allows the EM radiation beam to be emitted by the EM radiation source when the control device receives the transmit signal.

Another aspect of the disclosure is a method of eye protection for a participant using an EM radiation source capable of emitting an EM radiation beam suitable for use in an EM radiation-based procedure. The method includes operably arranging a control device relative to the EM radiation source. The control device is configured to control the emission of the EM radiation beam in response to a transmit signal. The method includes transmitting the transmit signal from a sensor unit to the control device. The sensor unit is operably disposed relative to a pair of protective eyewear and transmits the transmit signal only when the protective eyewear is properly worn by the participant.

Another aspect of the disclosure is the above-described method, wherein the EM radiation source includes a remote interlock connector, and wherein the method further includes matingly engaging the control device with the remote interlock connector, and controlling the emission of the EM radiation beam via the remote interlock connector.

Another aspect of the disclosure is the above-described method where the control device includes first and second modules and the method further includes matingly engaging the first module of the control device with the remote interlock connector. The first module is configured to control the emission of the EM radiation beam via the remote interlock connector in response to a control signal. The method includes receiving the transmit signal from the protective eyewear at the second module and in response generating a control signal. The method further includes providing the first module with the control signal via at least one of wired and wireless communication.

It is to be understood that both the foregoing general description and the following detailed description present embodiments of the disclosure, and are intended to provide an overview or framework for understanding the nature and character of the disclosure as it is claimed. The accompanying drawings are included to provide a further understanding of the disclosure, and are incorporated into and constitute part of this specification. The drawings illustrate various exemplary embodiments of the disclosure and together with the description serve to explain the principles and operations of the disclosure. The claims set forth below are incorporated into and constitute part of this specification.

### BRIEF DESCRIPTION OF THE DRAWINGS

FIG. 1A is a schematic diagram of a general example of the eyewear protection system according to the disclosure;

FIG. 1B is similar to FIG. 1A and includes more details pertaining to wireless and wired example embodiments that use control circuitry as part of the control device;

FIG. 1C is similar to FIG. 1B, except that the eyewear protection system is configured with radio-frequency identification (RFID) capability;

FIG. 1D is a schematic diagram of a portion of the EM radiation source, illustrating the optical path from the EM radiation unit to the EM radiation head, and showing an example embodiment where the control device includes an adjustable shutter disposed in the optical path;

FIG. 1E is a schematic diagram similar to FIG. 1A and that illustrates an example embodiment wherein the control device plugs into the remote interlock connector (port) of the EM radiation source;

FIG. 1F is similar to FIG. 1E and illustrates an example embodiment wherein the control device includes two modules, with one of the modules plugged into the port used as a remote interlock connector and the other module being in wired or wireless communication with the plugged in module;

FIGS. 2A through 2I illustrate various configurations of example embodiments of the protective eyewear and the sensor unit that senses whether the protective eyewear is properly worn by a participant;

FIG. 3 is a schematic diagram of a scene in which two participants (a user and a patient) are involved in an EM radiation-based medical procedure, where each of the participants is properly wearing their protective eyewear and the EM radiation beam is allowed to be emitted from the EM radiation head held and activated by the user;

FIG. 4 is similar to FIG. 3, and shows three participants (user, patient, observer); and

FIG. 5 is similar to FIG. 4, but shows an example where one of the participants (the observer) is not wearing protective eyewear, and wherein the EM radiation beam is not allowed to be emitted from the EM radiation head even though the user tries to activate the EM radiation source using an activating member.

### DETAILED DESCRIPTION

Reference is now made in detail to the embodiments of the disclosure, examples of which are illustrated in the accompanying drawings. Whenever possible, like or similar reference numerals are used throughout the drawings to refer to like or similar parts. Various modifications and alterations may be made to the following examples within the scope of the present disclosure, and aspects of the different examples may be mixed in different ways to achieve yet further examples. Accordingly, the true scope of the disclosure is to be understood from the entirety of the present disclosure, in view of but not limited to the embodiments described herein.

As used herein, the terms “protective eyewear” and “pair of protective eyewear” are used interchangeably and are each singular. In the description below, protective eyewear is considered as being properly worn when it substantially or completely blocks laser radiation from entering the eyes of the participant wearing the protective eyewear.

Also, the term “participant” includes anyone who is involved with an EM radiation-based procedure, and also includes anyone who might not be directly involved in the EM radiation-based procedure but that is in the “zone of danger” relative to the EM radiation source and that is at risk of eye damage if they are not wearing protective eyewear.

### Example Eye-Protection System Configuration

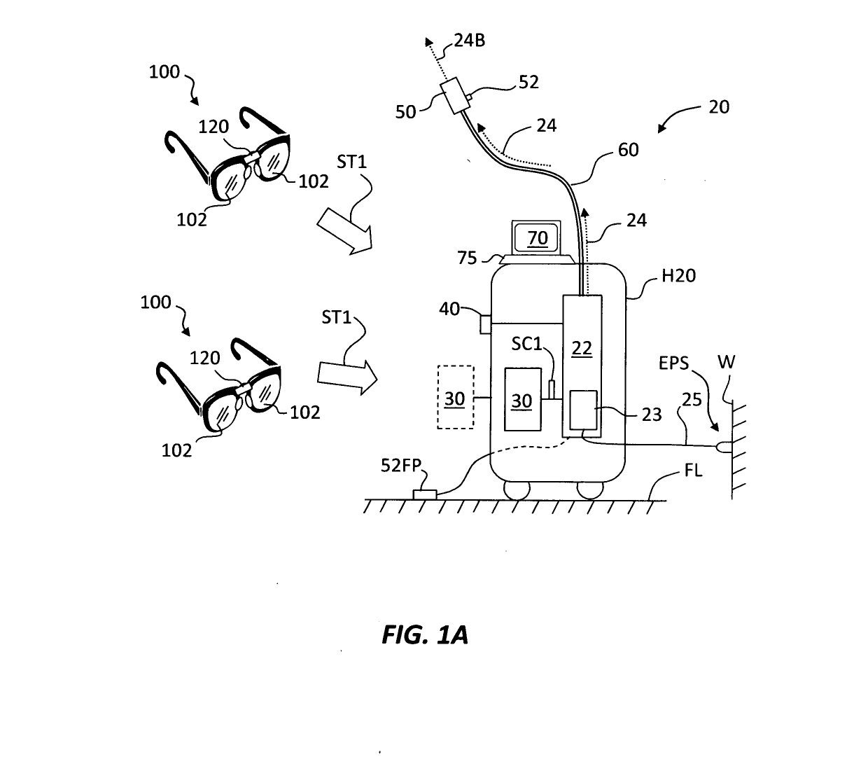

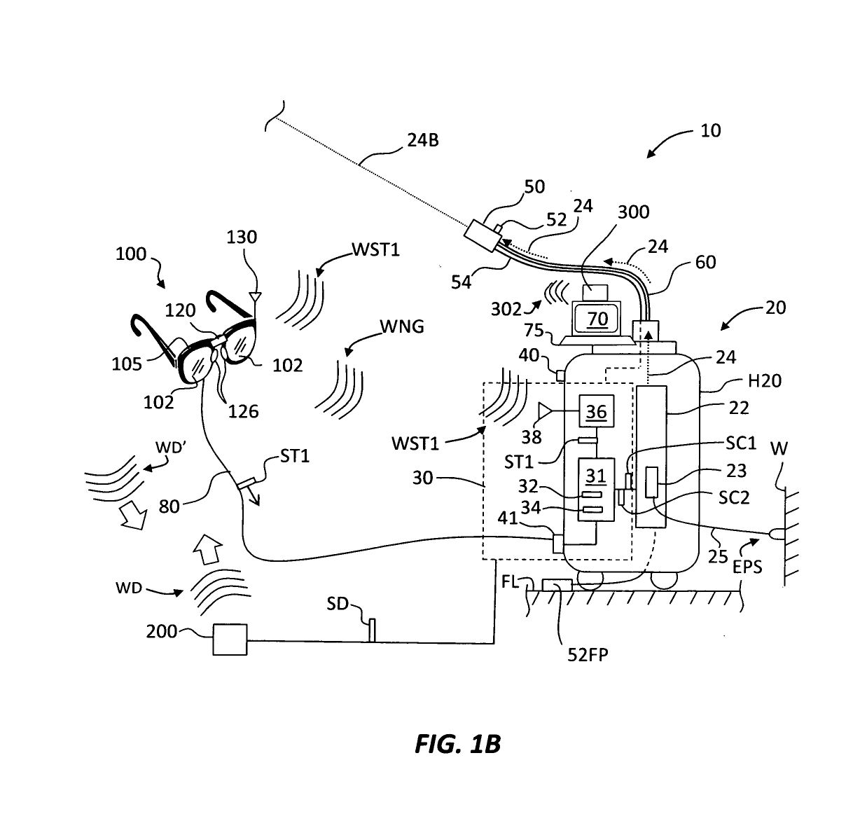

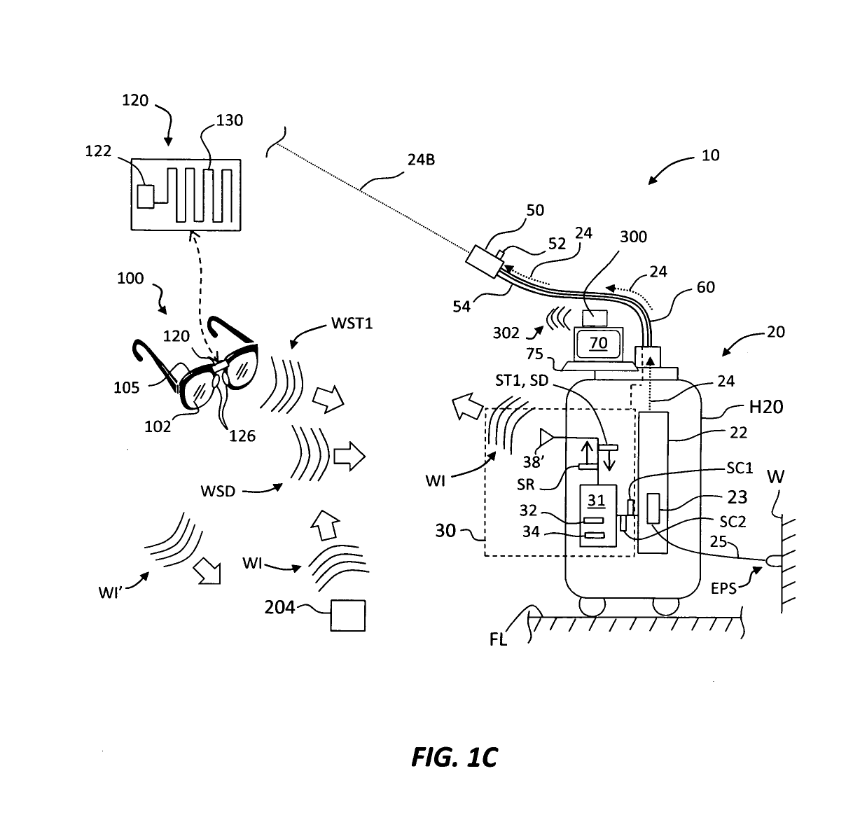

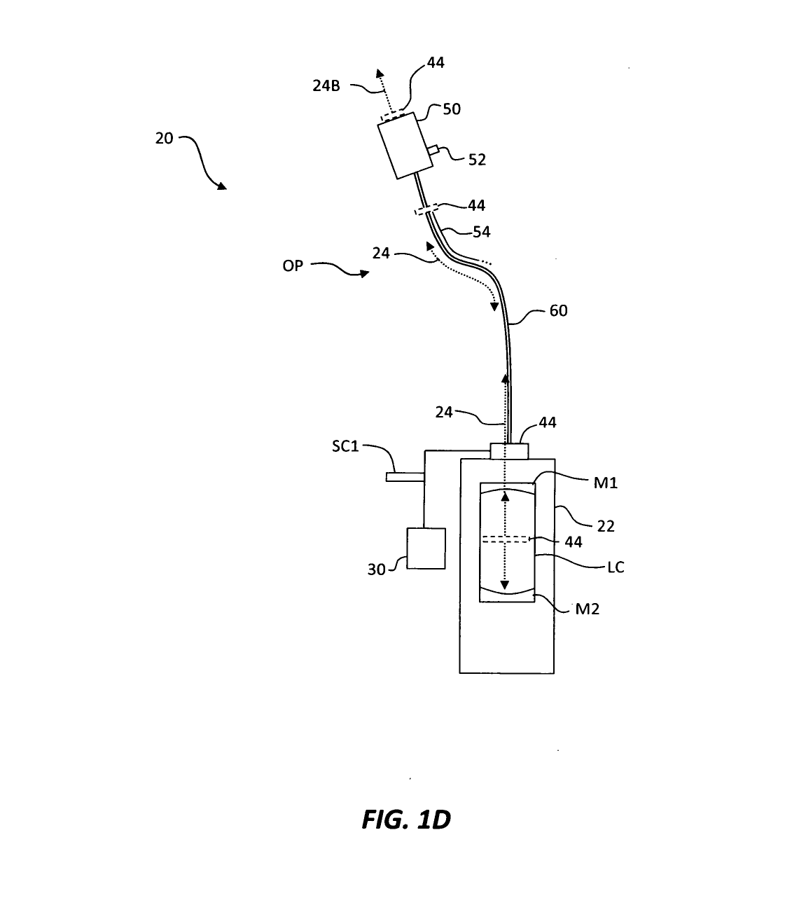

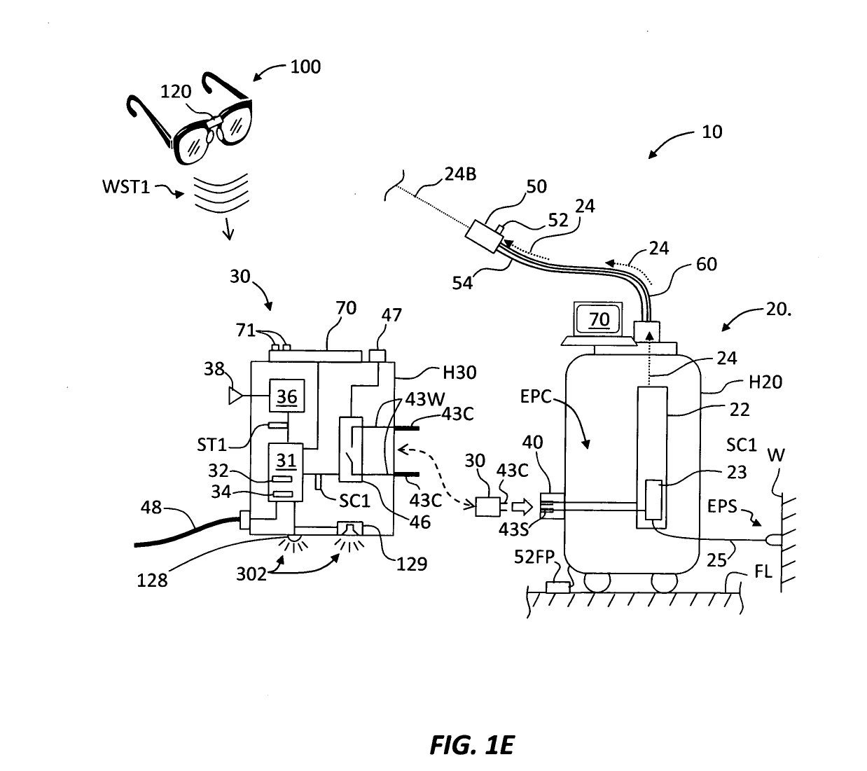

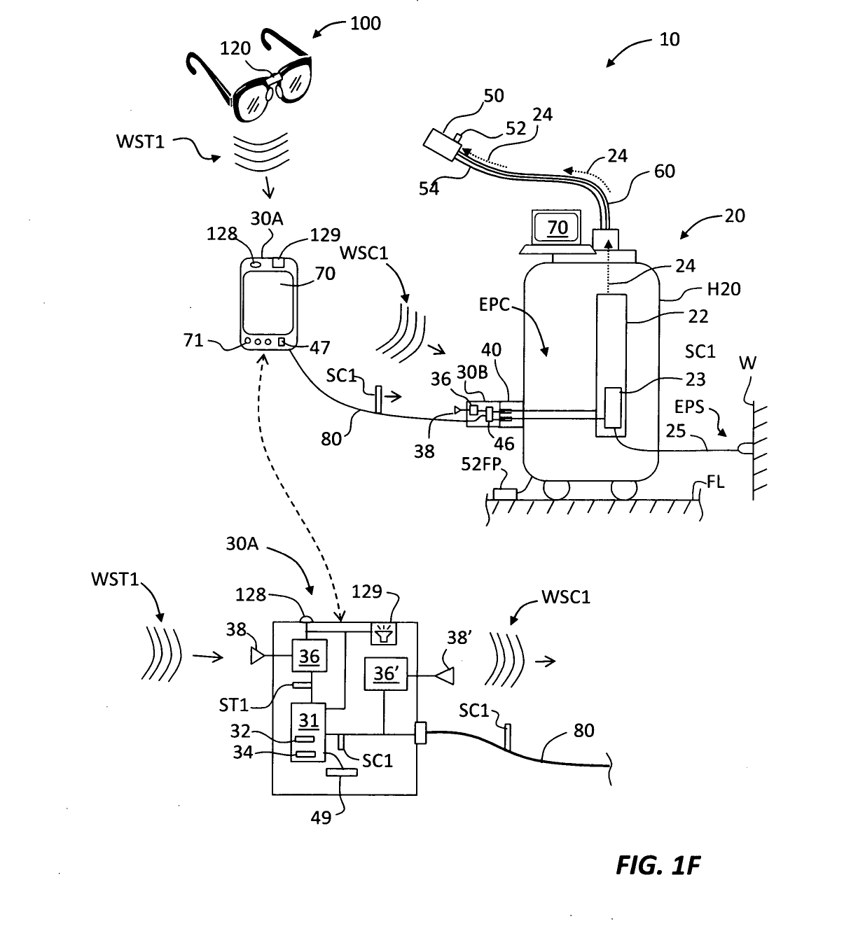

FIG. 1A is a schematic diagram of a general example embodiment of an eye-protection system (“system”) 10 according to the disclosure. FIGS. 1B and 1C are more detailed schematic diagrams of example systems 10 according to the disclosure.

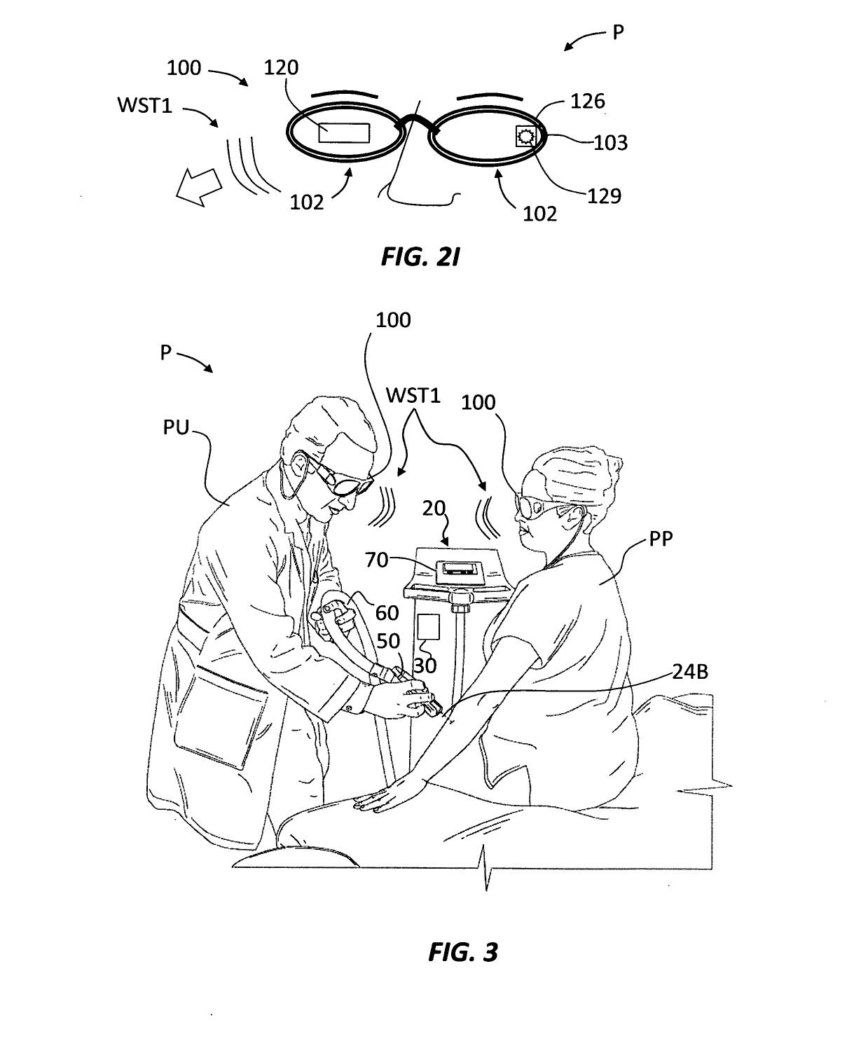

System 10 includes a control device 30 and at least one pair of protective eyewear 100 that includes a sensor unit 120. Two pairs of protective eyewear 100 are shown in FIG. 1A by way of illustration. The protective eyewear 100 is for use by participants P in an EM radiation-based procedure, as discussed in greater detail below in connection with examples shown in FIGS. 3, 4 and 5.

Control device 30 is configured to be operably arranged relative to an EM radiation source 20 and to control the emission of an EM radiation beam 24B from the EM radiation source. In an example, system 10 includes the EM radiation source 20. EM radiation source 20 may include an EM radiation unit 22 that generates EM radiation 24, and in an example, control device 30 is operably arranged relative to the EM radiation unit. EM radiation beam 24B generally refers to EM radiation emitted from EM radiation unit 20 while EM radiation 24 generally means EM radiation that has not yet been emitted from the EM radiation source.

In FIGS. 1A through 1C, participants P are not shown for ease of illustration, and protective eyewear 100 as shown therein is depicted as operating as if being worn by a participant P, such as shown for example in FIG. 3.

EM radiation source 20 can comprise any kind of source that emits EM radiation for use in an EM radiation-based procedure. An example EM radiation source 20 includes a housing H20. Examples of EM radiation source 20 include lasers or IPLs. IPLs are sometimes referred to as lasers but in actuality are non-laser sources of EM radiation that employ flash lamps. A laser-based EM radiation source 20 can include any one of a number of types of different lasers, such as those used in the medical profession to perform laser-based procedures. Example lasers include a CO₂ laser, a Nd-YAG laser, an erbium-based laser, a diode laser, a dye laser, an alexandrite laser, a ruby laser, an argon laser, a class 3B laser, a class 4 laser, a pulsed laser, and a continuous-wave laser.

EM radiation source 20 can be pulsed or continuous-wave. EM radiation source 20 is capable of emitting an EM radiation beam 24B. EM radiation beam 24B can have any wavelength such as radio wave, microwave, infrared, visible, ultraviolet, extreme ultraviolet and x-ray wavelengths, by way of example wavelengths.

In one example, control device 30 can be internal to EM radiation source housing H20, while in another example, it can be external to the EM radiation source housing, as described in the examples below (and as illustrated in phantom in FIG. 1A). EM radiation unit 22 is configured to generate EM radiation 24 that forms EM radiation beam 24B. An example EM radiation unit 22 includes a power supply 23 that provides electrical power needed for powering the EM radiation process that occurs within the EM radiation unit. EM radiation source 20 is shown by way of example as including a power cord 25 that is plugged into an external power supply EPS (e.g., an electrical socket) accessed in wall W and that provides electrical power to power supply 23.

An example EM radiation unit 22 includes a laser cavity for a laser-based EM radiation source 20. Another example EM radiation unit 22 is a flash lamp for a IPL-based EM radiation source 20. Control device 30 is configured to control the emission of EM radiation beam 24B from EM radiation source 20, as discussed in greater detail below.

In an example, EM radiation source 20 also includes an EM radiation head 50 optically coupled to EM radiation unit 22 by a cable 60 that in an example includes one or more optical fibers or other EM-carrying members (not shown) that carry EM radiation 24. EM radiation head 50 may be sized to be hand-held by a participant-user (“user”) PU (see, e.g., FIG. 3), such as a physician, physician's assistant, nurse, etc. It is noted here that not all EM radiation sources 20 have or even require an EM radiation head 50.

EM radiation head 50 is configured to receive EM radiation 24 from cable 60 and emit EM radiation beam 24B having a central or main wavelength λ_{B}. In an example, EM radiation beam can have a narrow wavelength band Δλ_{B} surrounding central wavelength λ_{B} such as in the case of some lasers, or can have a relatively wide wavelength band such as in the case of IPLs or other non-laser EM radiation sources.

In an example, system 10 includes an activation member 52 (e.g., switch, button, lever, etc.) that allows the user PU to control the emission of EM radiation beam 24B EM radiation source 20. In an example, activation member 52 resides on EM radiation head 50, while in another example, it resides elsewhere, e.g., as a foot pedal 52FP that can be activated by the foot of the user PU (see, e.g., FIG. 1A). System 10 can include multiple activation members 52, e.g., both a laser-head activation member and a foot pedal activation member.

In an example, EM radiation source 20 includes a port 40. In an example, port 40 comprises a remote interlock connector such as found on certain classes of lasers (e.g., class 3B and class 4 lasers) and that are required for door lockout connections (i.e., remote door interlocks). In an example, port 40 may be configured (e.g., with a splitter, not shown) to allow for multiple external wire connections or wireless connection to control device 30. An example of port 40 as comprising a remote interlock connector is discussed in greater detail below in connection with FIG. 1F.

In an example, EM radiation source 20 also includes a port 41 electrically connected to control circuitry 31. Port 41 may be, for example, configured to receive an electrical wire (e.g., a cable) 80 from protective eyewear 100, as shown in FIG. 1B, to provide a wire connection between the protective eyewear and control device 30.

In an example, system 10 includes a display 70 for displaying information relating to the operation system 10. In example, the displayed information relates to one or more operational states of system 10, such as the number of participants N_{P}, the status or mode of control unit 30, the type of EM radiation source used and its operating wavelength λB or wavelength band AA, the type of protective eyewear 100 required, how much power is in the EM radiation beam 24B, warning signs, messages for the participants P, etc. In an example, display 70 supports an interactive user interface (e.g., a touch screen) that allows the user PU to control the operation of system 10. In an example, display 70 is mounted on EM radiation source 20, but the display may located elsewhere, as illustrated in the different examples below. In an example, system 10 also includes an input device 75, such as a keyboard, for inputting information and commands in connection with controlling the various components of system 10 and the operation of the system as a whole.

### Example Control Devices

In an example, control device 30 performs its control function in response to receiving a wireless or wired (i.e., electrical) transmit signal WST1 or ST1 from protective eyewear 100. For example, in response to receiving a wireless or wired transmit signal WST1 or ST1, control device 30 provides EM radiation unit 22 with a control signal SC1 that prevents the EM radiation unit from emitting EM radiation beam 24B. For example, control signal SC1 may be provided to power supply 23 to stop electrical power from reaching the other parts of EM radiation unit 22 to stop the process that generates EM radiation 24. In another example described below in connection with FIG. 1F, control signal SC1 may be provided to a switch 46 of control device 30, wherein the switch cuts off electrical power at a location within EM radiation source 20 such at the EM radiation source is prevented from emitting laser beam 24B.

In an alternate example, control signal SC1 allows EM radiation source 20 to emit EM radiation beam 24B, and the absence of control signal SC1 prevents the laser from emitting the EM radiation beam. In the examples below, it is assumed for the sake of discussion that transmit signal ST1 and its corresponding control signal SC1 are used to allow EM radiation source 20 to emit EM radiation beam 24B unless stated otherwise.

In an example, EM radiation beam 24B has sufficient intensity to perform an EM radiation-based procedure. Thus, in one example where EM radiation beam 24B is prevented from being emitted by EM radiation source 20, a weaker EM radiation beam is allowed to be emitted that would not be considered dangerous to a participant P. Thus, in one example, no EM radiation beam 24B is allowed to be emitted from EM radiation source 20, while in another example, a “weak” EM radiation beam 24B is allowed to be emitted, wherein the weak EM radiation beam does not represent any substantial risk of eye-damage.

With reference to FIGS. 1B and 1C, an example control device 30 includes control circuitry 31. In a wireless configuration, control device 30 includes a wireless receiver 36 electrically connected to control circuitry 31. Wireless receiver 36 includes an antenna 38. Control circuitry 31 is configured (e.g., via software, firmware, or a combination thereof) to control the operation of EM radiation source 20, including allowing or preventing EM radiation beam 24B from being emitted from EM radiation source 20 in response to wireless or wired transmit signal WST1 or ST1.

In an example illustrated in FIG. 1B, sensor unit 120 generates a “no go” signal WNG that tells control device 30 not to allow the EM radiation source 20 to emit EM radiation beam 24B because protective eyewear 100 is not be properly worn. Sensor 120 terminates the transmission of the “no go” signal WNG when protective eyewear is properly worn and starts transmitting transmit signal WST1.

In an example, control circuitry 31 includes a processor unit (“processor”) 32 and a memory unit (“memory”) 34. In an example embodiment, processor 32 is or includes any processor or device capable of executing a series of software instructions and includes, without limitation, a general- or special-purpose microprocessor, finite state machine, controller, computer, central-processing unit (CPU), field-programmable gate array (FPGA), or digital signal processor. In an example embodiment, the processor is an Intel XEON or PENTIUM processor, or an AMD TURION or other in the line of such processors made by AMD Corp., Intel Corp. or other semiconductor processor manufacturer.

Memory 34 is operably coupled to processor 32. As used herein, the term “memory” refers to any processor-readable medium, including but not limited to RAM, ROM, EPROM, PROM, EEPROM, disk, floppy disk, hard disk, CD-ROM, DVD, or the like, on which may be stored a series of instructions executable by processor 32.

The methods of controlling the emission of EM radiation beam 24B from EM radiation source 20 and controlling the operation of system 10 in general may be implemented in various embodiments in a machine-readable medium (e.g., memory 34) comprising machine readable instructions (e.g., computer programs and/or software modules) for causing control circuitry 31 to perform the methods and operations described herein.

In an example, control device 30 is configured to control the emission of EM radiation beam 24B from EM radiation head 50. In this embodiment, an electrical wire 54 that connects control device 30 to EM radiation head 50 may be employed (see FIG. 1B).

FIG. 1D is a schematic diagram of a portion of EM radiation source 20 illustrating an optical path OP of EM radiation 24 from EM radiation unit 22 to EM radiation head 50. In one example, EM radiation unit 22 includes a laser cavity LC having mirrors M1 and M2. In an example, control device 30 includes a shutter 44 disposed in the optical path OP, which extends from mirror M2 to just beyond the output end of EM radiation head 50. Shutter 44 is electrically connected to control circuitry 31 (e.g., via wire 54) and can reside anywhere along the optical path OP (as illustrated by the various shutters 44 shown in phantom), including within laser cavity LC of a laser-based EM radiation unit 22. In an example, shutter 44 is configured to either at least partially block EM radiation 24 in the absence of control signal SC1 and to allow EM radiation source 20 to emit EM radiation beam 24B in response to receiving control signal SC1.

The example location of shutter 44 in optical path OP is shown as being between EM radiation unit 22 and EM radiation head 50. Shutter 44 may comprise for example a mechanical shutter (e.g., an electro-mechanical shutter) or an electro-optical shutter. Generally, shutter 44 can be any type of shutter that can be remotely activated to block EM radiation. Shutter 44 may also comprise a beam deflector that deflects EM radiation 24 from optical path OP, preferably into an EM radiation absorber (not shown). In an example, shutter 44 is configured to substantially attenuate EM radiation 24.

Protective Eyewear with Sensor Unit

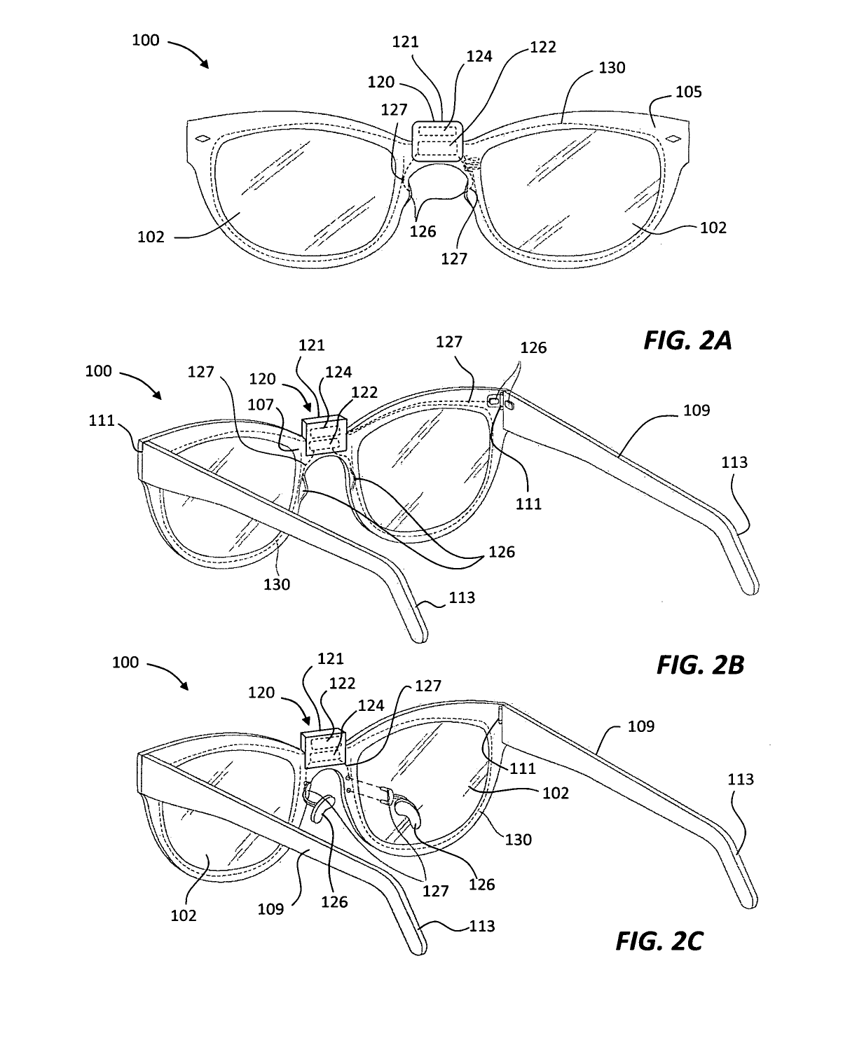

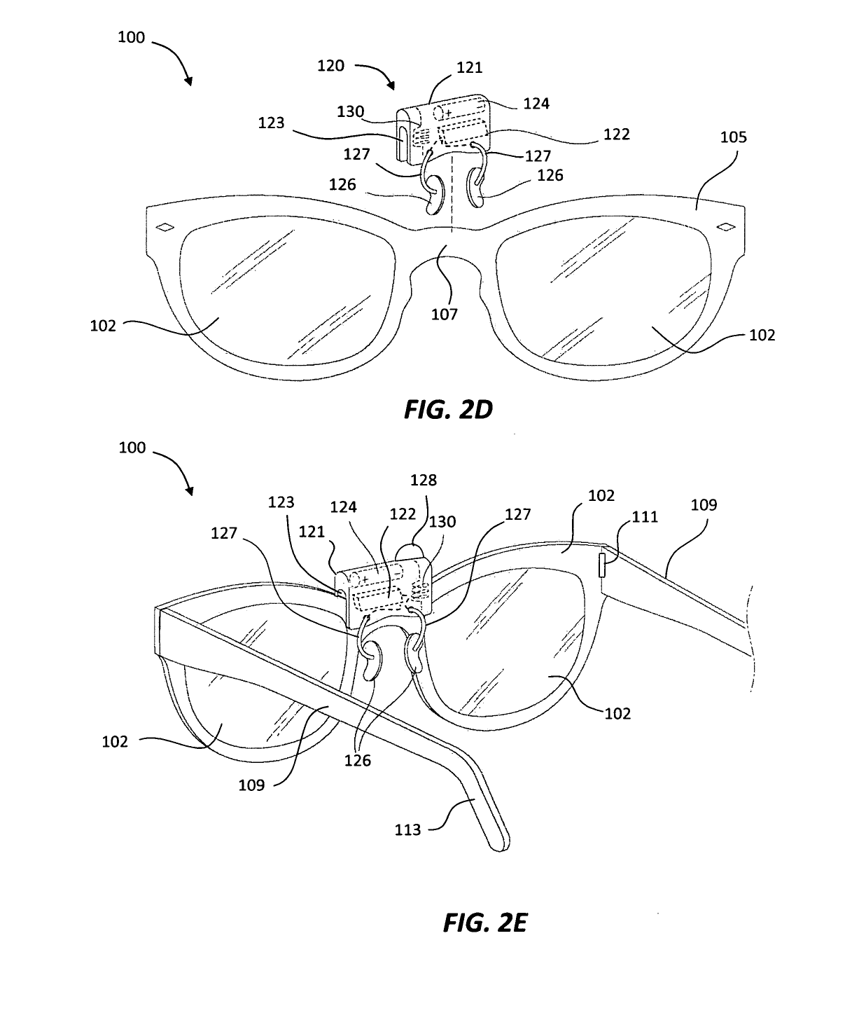

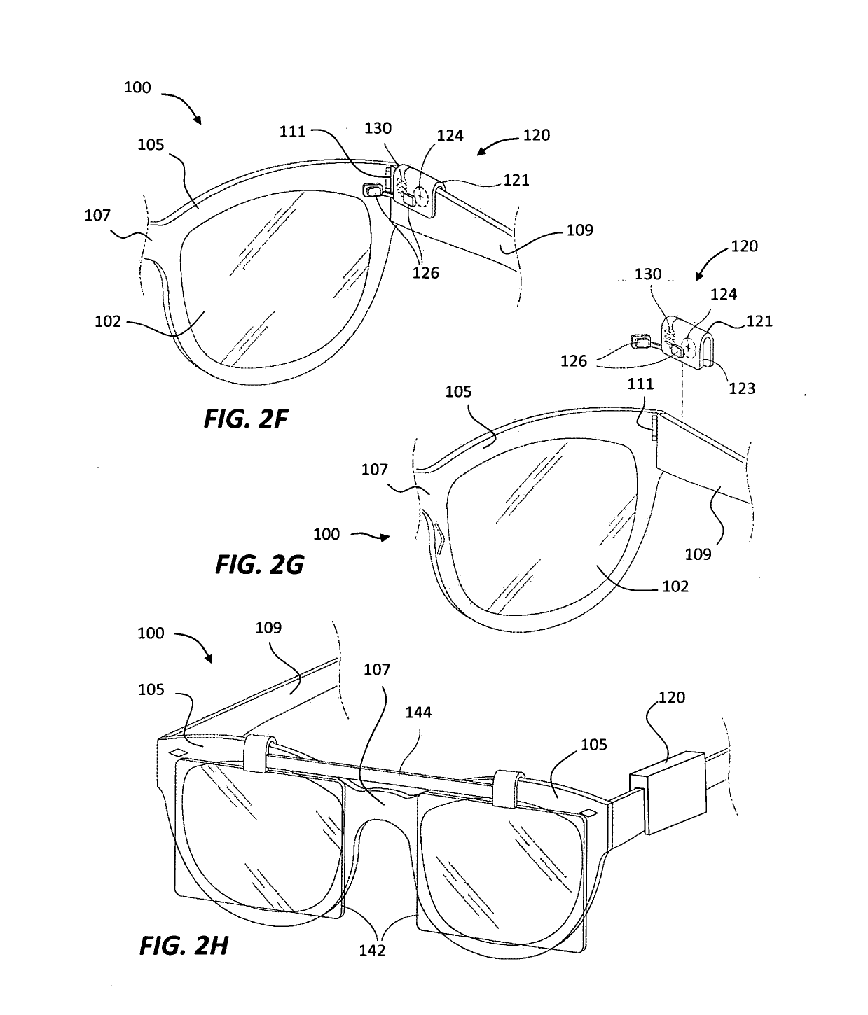

FIGS. 2A through 2I illustrate various example embodiments of protective eyewear 100 suitable for use in system 10 by participants P in an EM radiation-based procedure, as discussed in greater detail below. The examples below illustrate different configurations that allow for the generation of a wireless (i.e., electromagnetic) transmit signal WST1 or a wired (i.e., electrical) transmit signal ST1 to be generated and provided to the control device 30. Other configurations that fall within the spirit of the general concept of generating one or both of the wireless and wired transmit signal WST1 and ST1 are made possible by the various example described.

In an example, protective eyewear 100 is formed from conventional protective eyeglasses having lenses 102 that substantially block the EM radiation wavelength λ_{B} or wavelength band Δλ_{B}. Lenses 102 are supported by a frame 105. Frame 105 includes a bridge 107, and is connected to respective temples 109 by hinges 111. Temples 109 can have earpieces 113 to help secure the protective eyewear to the user.

Other forms of protective eyewear 100 such as goggles, extraocular and intraocular eye shields (see, e.g., FIG. 2I), and the like are contemplated, including types of protective eyewear that utilize a single lens 102 that protects both eyes. In some examples, protective eyewear is substantially opaque to a wide range of wavelengths and in particular is opaque to wavelength λ_{B} or wavelength band Δλ_{B} of EM radiation beam 24B.

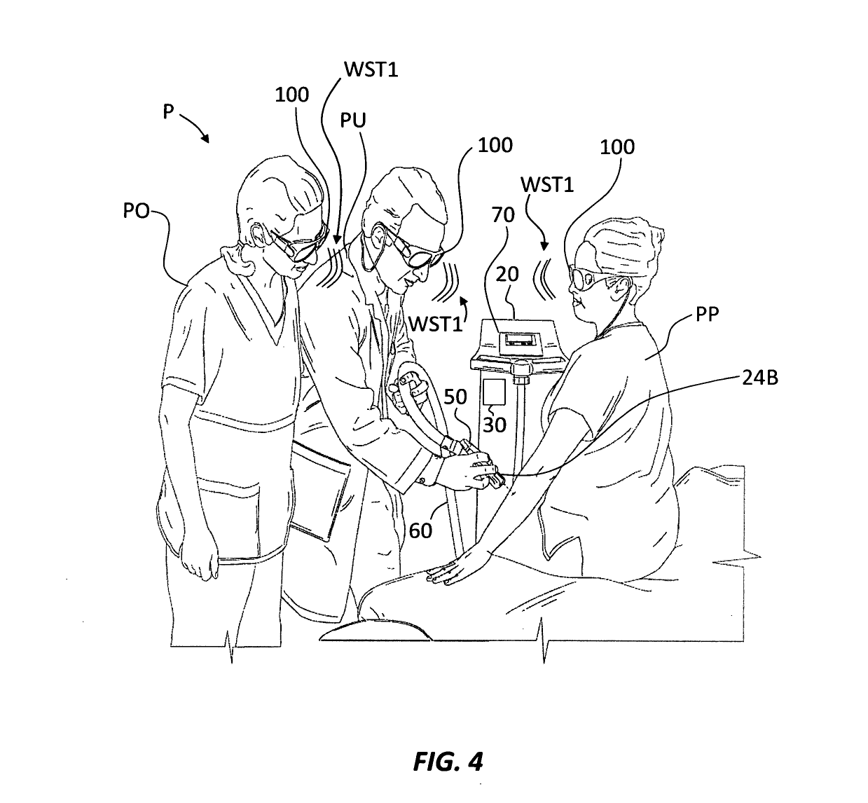

Protective eyewear 100 includes the aforementioned sensor unit 120. Sensor unit 120 is generally configured to sense when the protective eyewear is properly worn by a participant P (see e.g., FIGS. 3 and 4). In an example, sensor unit 120 includes a transmitter electronics chip 122 and a power source 124, such as a battery, electrically connected to the transmitter electronics chip. In an example, transmitter electronics chip 122 is electrically connected to antenna 130. Antenna 130 can have any one of a number of configurations, including being integrated into protective eyewear frame 105. In an example, antenna 130 is integrated into transmitter electronics chip.

In example embodiments of system 10, sensor unit 120 can be integrated into protective eyewear 100 or can be removably attached or clipped onto existing protective eyewear, as illustrated in the example embodiments of FIG. 2A through 21.

In an example, sensor unit 120 includes one or more activation elements 126 operably connected to transmitter electronics chip 122, e.g., via one or more connecting elements 127. The one or more activation elements 126 can be based on one or more of a variety of activating properties, such as temperature (e.g., body temperature), pressure, light, movement, heartbeat, electrical conductance, height above a floor FL (see e.g., FIG. 1A), and proximity to EM radiation source 20. In an example, the one or more connecting elements 127 are integrated into activation elements 126.

In one example, the one or more connecting elements 127 comprise wires that carry an electrical signal. In another example, the one or more connecting elements 127 comprise mechanical elements that communicate a mechanical force to transmitter electronics chip 122 and serve as a switch. In an example, transmitter electronics chip 122 is configured to be activated by the one or more activation elements 126 via the one or more connecting elements 127. In response, transmitter electronics chip generates an electrical transmit signal ST1 that is provided to antenna 130, which in response thereto generates wireless transmit signal WST1. As an alternative to or in combination with the formation wireless transmit signal WST1, the electrical transmit signal ST1 is transmitted via wire 80 to control device 30.

Sensor unit 120 is configured so that communication between the one or more activation elements 126 and the transmitter electronics chip 122 occurs when the participant P is properly wearing protective eyewear 100 (see e.g., FIG. 3). This in turn leads to transmitter electronics chip 122 and antenna 130 generating wireless transmit signal WST1 when the user is properly wearing the protective eyewear 100.

FIG. 1B also shows an alternative “wired” embodiment wherein electrical wire or cable 80 (dashed line) carries transmit signal ST1 from sensor unit 120 directly to control device 30 (e.g., to control circuitry 31) via port 41. In an example where electrical wire or cable 80 is used, the wire or cable can also carry electrical power to sensor unit 120.

In the wireless embodiment, wireless transmit signal WST1 is received by antenna 38 of wireless receiver 36 of control device 30. Wireless transmit signal WST1 is converted back into an electrical transmit signal ST1 by wireless receiver 36, which sends this signal to control circuitry 31.

As discussed above, in an example control circuitry 31 is configured to control the operation of EM radiation source 20, including activating and de-activating EM radiation unit 22, which is configured to generate EM radiation 24. In an example embodiment, control circuitry 31 receives electrical transmit signal ST1 and in response thereto provides a control signal SC1. Control signal SC1 serves to allow EM radiation unit 22 to generate EM radiation 24, e.g., by allowing electrical power to flow to the EM radiation unit. In an example, EM radiation unit 22 is held in an inactivated (e.g., a standby or “no laser output”) state in the absence of control signals SC1. In an alternate embodiment as discussed above, control signal SC1 serves to terminate the transmission of control signals SC2 to EM radiation unit 22 that maintain the EM radiation unit in an inactive state, i.e., the absence of control signals SC2 allows the EM radiation unit to generate EM radiation 24 (see FIG. 1B).

In some embodiments, if protective eyewear 100 is not properly worn by the user, control circuitry 31 causes display 70 to display a message that indicates EM radiation source 20 cannot be activated to generate EM radiation beam 24B until each of the participants P involved in the EM radiation-based procedure being performed is properly wearing protective eyewear.

When protective eyewear 100 is properly worn by all of the participants, control circuitry 31 changes the display message on display 70 to indicate that EM radiation source 20 can be activated by the user PU. Thus, in an example, even if all of the participants P are properly wearing their respective protective eyewear 100, the user still needs to activate EM radiation source 20, e.g., by engaging activation member 52. In an example, one of the participants P may be required to enter into system 10 the number N_{P} of participants prior to EM radiation source 20 being placed in an active mode where it can emit EM radiation beam 24B. In an example, a default number N_{P} of participants can be assumed by control device 30, such as two for a doctor and a patient in a medical procedure.

FIG. 1C is similar to FIG. 1B and illustrates an example embodiment wherein system 10 is configured with RFID capability. In particular, sensor unit 120 comprises an RFID tag (and thus is referred to as “RFID sensor unit 120” where applicable), wherein transmitter electronics chip 122 constitutes an RFID chip and antenna 130 constitutes an RFID antenna. Power supply 124 is optional, e.g., it can be used in some embodiments to provide an enhanced RFID transmit signal (defined below). In an example, RFID sensor unit 120 consists of transmitter electronics chip 122 and antenna 130 configured for RFID operation, and is in the form of an RFID tag, i.e., a thin, planar chip.

In the example illustrated in FIG. 1C, EM radiation source 20 of system 10 has its control circuitry 31 configured to generate electrical RFID signals SR and provide them to RFID antenna 38′, which in response generates a wireless RFID interrogation signal WI. RFID interrogation signal WI is received by RFID sensor unit 120, and the energy from this interrogation signal is used to drive transmitter electronics chip 122. In response, transmitter electronics chip 122 imparts information onto a reflected RFID signal WST1 transmitted (reflected) by antenna 130. RFID signal WST1 is referred to as “RFID transmit signal” in this embodiment because it is analogous to the transmit signal formed by the non-RFID embodiment of sensor unit 120. The information in RFID transmit signal WST1 indicates whether protective eyewear 100 is being worn properly by the particular participant P. In an alternative example embodiment, the source of wireless RFID interrogation signal WI can be elsewhere on EM radiation source 20 or elsewhere in the vicinity of the EM radiation source, such as illustrated in FIG. 1C by separate interrogation source 204.

In an example, when protective eyewear 100 is properly worn, the one or more activation elements 126 allow RFID sensor unit 120 to provide RFID transmit signal WST1 that indicates the protective eyewear is being worn correctly. However, when protective eyewear 100 is not being worn or is being worn incorrectly, the one or more activation elements 126 are not activated and RFID sensor unit 120 generates a default RFID signal WSD that indicates to control device 30 that it is not OK to allow EM radiation source 20 to emit EM radiation beam 24B.

When protective eyewear 100 is being worn correctly so that interrogation signal WI elicits an RFID transmit signal WST1 from RFID sensor unit 120, the RFID transmit signal is received by RFID antenna 38′ (or other antenna, not shown) of control device 30. In response, RFID antenna 38′ generates an electrical transmit signal ST1 that travels to control circuitry 31. Transmit signal ST1 indicates that it is OK for control circuitry 31 to allow EM radiation source 20 to emit EM radiation beam 24B. In the embodiment where RFID sensor unit 120 generates the default reflected RFID signal WSD, then the corresponding transmit signal SD indicates that it is not OK for control circuitry 31 to allow EM radiation source 20 to emit EM radiation beam 24B. This in turn can trigger an informational signal 302, as introduced and discussed below, that indicates the status of the EM radiation source 20 as not being able to emit EM radiation beam 24B.

FIG. 1E is a close-up view of control device 30 and EM radiation source 20 and illustrates an example embodiment where control device 30 is configured to plug into port 40. Here, port 40 is shown by way of example as being in the form of a remote interlock connector having two electrical sockets 43. Electrical sockets 43S are shown by way of example as being part of an electrical power circuit EPC formed by power supply 23 of EM radiation unit 22.

The example control device 30 of FIG. 1E includes a housing H30 and two electrical contacts 43C configured to matingly engage electrical sockets 43S when the control device 30 is plugged into port 40. It is noted here that remote interlock connector can have different configurations (e.g., such as on class 3B and class 4 lasers), and may have more than two electrical contacts 43C such as are shown by way of example. In an example, control device 30 includes the aforementioned control circuit 31, and wireless receiver 36 with antenna 38.

The example control device 30 can include display 70 (e.g., a microdisplay) that optionally includes user interface elements 71 (pushbuttons, dials, switches, etc.). Control device 30 also includes a switch 46 electrically connected to control circuitry 31 and to wires 43W that lead to electrical contacts 43C. Control device 30 includes an override switch 47 electrically connected to switch 46 and that can be manually engaged to control power switch 46. In an example, a power cable 48 is connected to control device 30 to provide power to the control device. In another embodiment, control device 30 receives electrical power from EM radiation source 20 via port 40 or from a battery 49. In an example, switch 46 is configured to remain closed for a select amount of time and then re-open.

Control device 30 of FIG. 1E is also shown as having an indicator light 128 and a speaker 129 that can respectively generate a visual and an audible informational signals 302. In an example the visual and audible signals 302 represent at least one operational state of system 10, or at least one operation state of at least one of protective eyewear 100, control device 30 and EM radiation source 20.

In an example of the operation of control device 30 of FIG. 1E, in response to transmit signal ST1, control circuitry 31 generates control signal SC1 that controls the operation of switch 46. In particular, in the absence of transmit signal ST1, power switch 46 remains in the open state (as shown), which opens the electrical power circuit EPC shown as being associated with EM radiation unit 22 and power supply 23. In this open switch state, EM radiation unit 22 cannot receive electrical power and so cannot generate EM radiation 24. In general, electrical power circuit EPC can have any configuration that provides power for EM radiation source 20 that results in the generation of EM radiation beam 24B.

When transmit signal ST1 is received, control signal SC1 is generated and is provided to switch 46, which closes switch 46 and closes the electrical power circuit EPC to establish the flow of electrical power in the electrical power circuit. In this closed switch state, EM radiation unit 22 receives electrical power so that the EM radiation unit can generate EM radiation 24. As noted above, in the example where switch 46 automatically re-opens after a select amount of time, a new control signal SC1 is needed to close the switch or to keep the switch closed.

It is noted here that power supply 23 is shown by way of example as one source of electrical power for EM radiation unit 22, and that the electrical power circuit EPC can be defined in relation to any power source that provides electrical power to EM radiation unit 22, including from external power supply EPS received via power cable 25.

### Control Device Having First and Second Modules

FIG. 1F is similar to FIG. 1E and illustrates an example embodiment for system 10 wherein an example control device 30 includes first and second modules 30A and 30B. First module 30A is shown by way of example in the form of a hand-held unit that includes essentially the same components as that of the control device 30 of FIG. 1E but without switch 46. First module 30A also includes a wireless transmitter 36′ that includes a transmitter antenna 38′. In an example, first module 30A is in the form of a hand-held electronic device, such as an i-POD® or i-PAD® device available from Apple, Inc. First module 30A can be thought of as a remote interface device for control device 30A.

Second module 30B includes switch 46, along with a wireless receiver 36 electrically connected to the switch, with the wireless receiver having a wireless antenna 38. In an example, second module 30B can include control circuitry (not shown).

First module 30A and second module 30B are also shown as being electrically connected by a wire 80, as an alternate to or for use in combination with the wireless configuration. Thus, the first and second modules 30A and 30B may be in at least one of wired and wireless communication. It is also noted that in various example embodiments, the different components that make up control device 30 can be distributed between the first and second modules 30A and 30B, and that FIG. 1F illustrates one example configuration of the distribution of different components. For example, in one embodiment, first module 30A may simply serve to relay wireless or wired transmit signal WST1 or ST1 from sensor unit 120 to second module 30B, which then processes the transmit signal to generate control signals ST1.

In an example of the operation of the wireless embodiment of system 10 of FIG. 1F, first module 30A receives wireless transmit signal WST1 from protective eyewear 100. Wireless transmit signal WST1 is processed by first module 30A as described above to generate control signal SC1, which is provided to wireless transmitter unit 36′. Wireless transmitter 36′ and transmitter antenna 38′ generate a wireless control signal WSC1 that is transmitted to second module 30B. Wireless control signal WSC1 is received by receiver antenna 38 of wireless receiver unit 36 of module 30B, and the wireless receiver unit converts the wireless control signal into an electrical control signal SC1. Electrical control signal SC1 is provided to switch 46 and serves to close switch 46, thereby completing the electrical power circuit EPC.

In the operation of the wired embodiment of system 10 of FIG. 1F, first module 30A receives wireless transmit signal WST1 from protective eyewear 100. Wireless transmit signal WST1 is processed by first module 30A as described above to generate electrical control signal SC1, which sent over wire 80 to second module 30B. Electrical control signal SC1 is provided to switch 46 and serves to close switch 46 and to establish the electrical power circuit EPC.

### Protective Eyewear and Sensor Unit Example Configurations

FIG. 2A is a front-on view and FIG. 2B is a rear elevated view of an example glasses-type of protective eyewear 100 wherein sensor unit 120 clips onto or is integrated into frame 105 at bridge 107. Sensor unit 120 includes a sensor unit housing 121 that contains transmitter electronics chip 122 and power source 124 (which is optional in the RFID sensor unit configuration). Antenna 130 is shown as being supported by frame 105.

In an example, the one or more activation elements 126 are incorporated into the nose-support portion of frame 105, and are electrically connected to transmitter electronic chips by conducting connecting elements 127 (e.g., wires). In an example, the one or more activation elements 126 are activated by at least one of pressure and heat from a participant P's nose when protective eyewear is properly worn. FIG. 2C is similar to FIG. 2B and is an exploded view of the one or more activation elements 126 in the form of nose support members.

FIG. 2D is a front-on view and FIG. 2E is a rear elevated view of an example glasses-type of protective eyewear 100 illustrating an example embodiment where sensor unit housing 121 includes a groove 123 sized to receive bridge 107 of frames 105. This configuration of sensor unit 120 allows the sensor unit to be added directly to an existing frame 105. Antenna 130 is shown incorporated into sensor unit housing 121. The one or more activation elements 126 are also shown as being directly supported by sensor unit 120 rather than by frame 105.

In an example, protective eyewear 100 includes an indicator light 128 (see FIG. 2E, introduced and discussed below) that can provide an indication of battery power and that can also indicate whether EM radiation source 20 can emit EM radiation beam 24B, e.g. a red indicator light to indicate that sensor unit 120 on protective eyewear 100 is not activated, and a green indicator light to indicate that the sensor units on the protective eyewear have been activated. Generally, indicator light 128 is used to indicate at least one operational state of protective eyewear 100.

FIG. 2F and FIG. 2G are partial elevated rear views that illustrate an example embodiment where sensor unit housing 121 includes groove 123 sized to accommodate a portion of temple 109 adjacent hinge 111. Activation members 126 are in the form of contact pads disposed so that they come with the sides of the participant's head to be activated.

FIG. 2H is a front-elevated view of an example of protective eyewear 100 that includes filter lenses 142 respectively arranged in front of lenses 102. Filter lenses 142 are supported on protective eyewear 100 by a filter support member 144 that clips onto frame 105. Filter lenses 142 can also be configured as a single filter lens that covers both lenses. In another embodiment, filter lenses 142 are configured to eliminate or change the color of informational light 302 that is emitted before EM radiation beam 24B is emitted from EM radiation source 20.

In an example, filter lenses 142 are configured to make an otherwise visible informational signal 302 of a certain color (wavelength) disappear or change color. Filter lenses 142 can be attached to existing protective eyewear 100 or built into existing protective eyewear from other systems.

FIG. 2I is a front-on view of an example of protective eyewear 100 in the form of an opaque eye shield being worn by a participant P. This protective eyewear 100 is shown as having its one or more activation elements located at the outer rim 103 of each eye shield “lens” 102, which is opaque to the operating wavelength λ_{B} or wavelength band Δλ_{B}. The one or more activation elements can operate using, for example, at least one of temperature and pressure.

In an example, a small indicator element 129 such as a screen or light (e.g., LED) is provided on one of the eye shield lenses 102 to visually indicate whether or not the eye shield is properly in place over the eyes of participant P. In one example embodiment, protective eyewear 100 is in the form of an extraocular eye shield, while in another example embodiment protective eyewear 100 is in the form of an intraocular eye shield.

### Example Methods of Operation

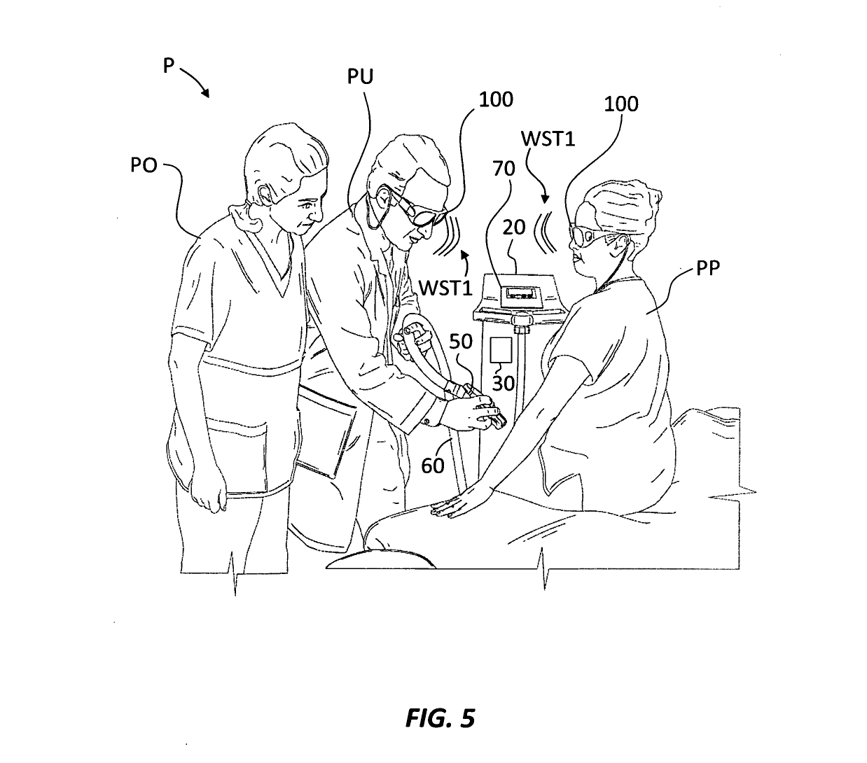

FIGS. 3 through 5 illustrate examples of a EM radiation-based procedure in the form of a medical procedure that includes a number N_{P} of participants P, including a user PU that controls EM radiation source 20, and a patient PP. Each participant P needs to wear protective eyewear 100 during the EM radiation-based procedure or risk eye injury. FIG. 4 and FIG. 5 additionally show an observer participant PO.

With reference to FIG. 3, in an example method of operation of system 10 the user PU and patient PP put on their protective eyewear 100. This serves to activate the one or more activation elements 126 operatively arranged on each pair of protective eyewear 100. In response, in each pair of protective eyewear 100, the one or more activation elements 126 communicate with sensor unit 120 to cause the respective sensor units 120 to generate corresponding transmit signals ST1. In one example, each transmit signal ST1 is converted to wireless transmit signal WST1 that travel to control device 30. In another example, the transmit signal ST1 travels to control device 30 as a non-wireless (electrical) signal ST1 via electrical wire or cable 80. A combination of wired and wireless transmit signals ST1 and WST1 can also be used.

The transmit signals (WST1 and/or ST1) are received by control device 30. In an example, control device 30 is configured to compare the number N_{T} of transmit signals to a number N_{P} of participants P. If control device 30 is unsure of the participant number N_{P}, it can audibly or visually ask user PU to enter the number N_{P} of participants P, e.g., at a user interface provided on display 70. Once the number N_{T} of transmit signals matches the participant number N_{P}, control device 30 allows EM radiation source 20 to emit EM radiation beam 24B when user PU activates the EM radiation source. In an example, as long as protective eyewear 100 is properly worn by all the participants P, EM radiation source 20 will be allowed to emit EM radiation beam 24B when the user PU activates the EM radiation source. In another example, EM radiation source 20 is allowed to emit EM radiation beam 24B for a select amount of time after which control device places the EM radiation source in an inactive mode (e.g., by automatically opening switch 46, as discussed above) if it does not receive a transmit signal ST1. In the case where sensor unit 120 is the aforementioned RFID sensor unit, interrogation signal SI is periodically transmitted to protective eyewear 100, which causes the RFID sensor unit to periodically generate RFID transmit signals ST1 when the protective eyewear is properly worn.

FIG. 5 is similar to FIG. 4 and illustrates an example where the observer participant PO is not wearing protective eyewear 100, and the user PU attempts to activate EM radiation source 20. Because observer participant PO is not wearing protective eyewear 100, no EM radiation beam 24B is generated by EM radiation source 20.

It is emphasized here that without the use of the systems and methods described herein, any other type of lockout, such as door-based lockouts, would not be an effective safety measure in the situation shown in FIG. 5. That is to say, an EM radiation source 20 configured with a door-based lockout still presents a substantial risk of eye injury to the observer PO in FIG. 5 because she forgot to wear protective eyewear even if the door to the room is closed.

In an example, control device 30 frequently samples the number of transmit signals ST1 it receives (e.g., once per second or many times per second) to ensure that protective eyewear 100 is sending transmit signals WST1 or ST1 indicative of the participants P each wearing their protective eyewear. If a certain amount of time lapses where no transmit signal is received by the control device from the protective eyewear, the control device renders the EM radiation source unable to emit EM radiation beam 24B.

In an example, different pairs of protective eyewear 100 generate different transmit signals ST1 (e.g., the transmit signals each have a different signal feature, such as a slightly different frequency) so that control device 30 can distinguish between the different protective eyewear. Also in an example, the different transmit signals ST1 include information about the particular laser to which protective eyewear 100 is suited. This can be used to prevent emission of EM radiation beam 24B if the protective eyewear for the corresponding EM radiation source 20 is not being properly worn (e.g., protective eyewear for a different EM radiation source is worn by mistake).

### Other Example System Configurations

In an example, system 10 functions on the basis that the participants P in the EM radiation-based procedure need to be properly wearing protective eyewear 100 so that sensor unit 120 can generate either a wireless transmit signal WST1 or a wired transmit signal ST1 that causes the control device 30 to control the emission of EM radiation beam 24B from EM radiation source 20 (e.g., prevent the emission of EM radiation beam 24B in the absence of a transmit signal).

In an example, at least one of a wireless and wired transmit signal WST1 and ST1 is transmitted each time the user PU operates EM radiation source 20, e.g., via activating member 52 on EM radiation head 50 or foot pedal activating member 52FP. In another example, only a single transmit signal is initially sent to trigger the initial activation of EM radiation source 20, and the EM radiation source can remain active for a select time period thereafter. For example, once EM radiation source 20 is successfully activated by the transmit signal, then the EM radiation source is allowed to remain active for select time period suitable for the particular EM radiation-based procedure being carried out. For example, for a laser-based medical procedure, an example suitable activation time is a few seconds. After the suitable activation time, the control unit 30 will have to receive another transmit signal to allow the EM radiation source 20 to remain in the active operational state.

In one example, system 10 is configured so that the transmit signals from the protective eyewear from the user PU are the only transmit signals processed by control unit 30 even if there are more participants. This embodiment relies on the user PU to ensure that all participants are wearing their protective eyewear.

In another example, system 10 is configured so that transmit signals need to be received from the operator PU and at least one other participant P.

In another example, system 10 is configured so that transmit signals need to be received from all of the participants P. In an example, the number of participants N_{P} can be entered into system 10 manually or wireless via a remote wired or wireless interface device (e.g., on a remote display such as on display 70 of first control device module 30A) so that the control device can keep track of the number of transmit signals ST1 it needs to receive before allowing EM radiation source 20 to emit EM radiation beam 24B.

In many cases, just two participants P will be present (i.e., N_{P}=2), namely a trained medical person as the user PU and a patient PP. In this case, system 10 may be configured to ensure that both the trained medical person and the patient are wearing their protective eyewear 100 before EM radiation source 20 can emit EM radiation beam 24B.

In an example embodiment illustrated in FIG. 1B, system 10 further includes a detector unit 200 configured to automatically detect the number N_{P} of participants P in a given space around the system, e.g., within a room where system 10 resides or in a select area around where the EM radiation based procedure is being carried out. Detector 200 can be anywhere in the given space, and is preferably positioned in a location where all of the participants P can be detected via a detection signal WD emitted by the detector. In an example, in response to receiving information from the returned detector signal WD', detector unit 200 provides an electrical detector signal SD to control device 30, wherein the detector signals includes information about the number N_{P} of participants P. In an example, detector unit 200 is configured to sense motion. In some embodiments, detector unit 200 can be integrated into a remote wired or wireless interface device, e.g., into first control device module 30A.

Detector unit 200 can be programmed to sense participants P within a certain radius of or distance from EM radiation source 20 or EM radiation head 50. If system 10 is already in operation and detector 200 detects a new participant P, then detector signal SD can be used by control device 30 to prevent EM radiation beam 24B from being emitted from EM radiation source 20 until the new participant either leaves or dons protective eyewear 100. Thus, in an embodiment the number N_{P} of participants must equal the number of transmit signals ST1 from the protective eyewear 100 appropriate for the particular EM radiation source 20.

System 10 may also have redundancy whereby the user PU is required to enter the number of participants N_{P} directly into EM radiation source 20 (e.g., at the user interface of display 70 or at the remote wired or wireless interface of control device 30), and control device 30 verifies the entered number N_{P} with the corresponding number from detector 200 and from the transmit signals ST1 received from the protective eyewear. In an example, display 70 displays the number N_{P} of participants and asks the user PU to confirm the number of participants detected.

FIG. 1B and FIG. 1C illustrate an example embodiment wherein system 10 includes an informational signal unit 300 that generates an informational signal 302 representative of at least one operational state of system 10. For example, informational signal 302 can indicate that one or more participants P is/are not wearing protective eyewear 100. Informational signal 302 may be a visible signal, an audio signal or both. Informational signal unit 300 may also include separate visual and audio portions that reside in spaced-apart housings, and informational signal unit is shown as contained in a single housing by way of example. The audio signal 302 may be an alarm sound or a voice sound that provides appropriate warning language. In an example, informational signal unit 300 is located on EM radiation head 50, EM radiation source 20 or in another location within the room and operably coupled (e.g., by a wire or wirelessly) to control unit 30.

In an example, before EM radiation source 20 is activated to emit EM radiation beam 24B, informational signal unit 300 generates informational signal 302, such as a red light that is readily visible to the user PU. This type of visible informational signal 302 can be of sufficient intensity to light up the space or area in which the participants reside (e.g., the space within a room), can be a strobe light or blinking light. In an example, informational signal 302 can be emitted from EM radiation head 50 and shine on a surface (e.g., an area of patient PP to be treated) and include highlighted words such as “STOP” or “NEED GOGGLES.”

In an example, if user PU is properly wearing his or her protective eyewear 100, he or she does not see visible informational signal 302, either because it is not generated or because the wavelength of the visible informational signal is filtered by the protective eyewear 100 (e.g., by filter lenses 142 and/or by lenses 102). Consequently, if the user PU sees a visible informational signal 302, it is a clear indication that he or she must put on their protective eyewear 100 to be able to safely activate EM radiation source 20.

In an example, informational signal unit 300 generates a visible informational signal 302 having a wavelength λ_{W} that is filtered by lenses 102 of protective eyewear 100. For example, visible informational signal 302 can have a red wavelength λ_{W} associated with an EM radiation source 20 that generates an EM radiation beam 24B having an infrared wavelength of λ_{B}=1064 nm. Lenses 102 of protective eyewear 100 are designed, e.g., via optical coatings, using a select lens material for lenses 102, or a combination thereof, to filter the red wavelength λ_{W} of visible informational signal 302 as well as the EM radiation beam wavelength λ_{B}=1064 nm.

In some instances where the EM radiation-based procedure is to be carried out using multiple EM radiation sources 20, it is possible for user PU to inadvertently put on protective eyewear 100 for the wrong EM radiation source. For example, if a room includes first and second EM radiation sources 20 in the form of lasers operating at respective wavelengths λ₁ of 1064 nm and λ₂ of 1540 nm, it may be that the first protective eyewear 100 for the first laser is not sufficiently protective for use with the second laser.

Thus, in an example embodiment, protective eyewear 100 is configured to work with a select type of EM radiation source 20 through an appropriately configured control unit 30. This can serve to prevent the user PU from wearing the wrong protective eyewear 100 during the EM radiation-based procedure. For example, first protective eyewear 100 is configured to only activate first EM radiation source 20 and not second EM radiation source 20.

This issue can also be addressed using informational systems 300 on the different EM radiation sources 20. For example, system 10 may include first and second warning systems 300 for first and second EM radiation sources 20, respectively, where the first and second warning systems generate respective visible informational signals 302 with different wavelengths λ_{W}, say red and blue. The first and second protective eyewear 100 are configured to only filter visible informational signals 302 having a wavelength λ_{W} of the corresponding EM radiation source 20 and not the other EM radiation source. Thus, if the user PU puts on protective eyewear 100 for second EM radiation source 20 but tries to operate the first EM radiation source, he or she will see the red warning light 302 since the red wavelength is not blocked by the protective eyewear for the second EM radiation source 20, e.g. the protective eyewear 100 for second EM radiation source filters blue light.

It is often the case that user PU of system 10 is responsible for making sure that the other participants P are each wearing their protective eyewear 100. Thus, in an example, when user PU tries to activate EM radiation source 20 and not all of the participants P are wearing protective eyewear 100, informational signal unit 300 sends out an audible informational signal 302 in the form of recorded message reminding all participants P that everyone needs to be wearing eye protection at this point. An example sample recorded message is “Everyone in this room must be wearing protective eye goggles.” In an example, this message is broadcast prior to EM radiation source 20 being placed in an active mode where it is capable of generating EM radiation beam 24B.

In an example of system redundancy, display 70 includes the aforementioned user interface that allows the user PU to manually place EM radiation source 20 in an active mode by confirming that all participants P are wearing protective eyewear 100.

1. An eye protection system for a participant in an EM radiation-based procedure that uses an electromagnetic (EM) radiation source capable of emitting an EM radiation beam and that includes a remote interlock connector, comprising:
a control device configured to matingly engage the remote interlock connector and control the emission of the laser beam via the remote interlock connector in response to a wireless transmit signal; and
a protective eyewear having a sensor unit, the sensor unit being operable to transmit the transmit signal to the control device only when the protective eyewear is properly worn by the participant.

2-4. (canceled)

5. The system of claim 1, wherein the control device comprises:
a first module configured to perform said mating engagement of the remote interlock connector and said control the emission of the EM radiation beam in response to a control signal or in response to said transmit signal; and
a second module that receives the wireless transmit signal from the protective eyewear and that is configured to provide the first module with either the control signal or the transmit signal in response to the second module receiving the transmit signal.

6. The system of claim 1, further comprising the EM radiation source, with the control device being matingly engaged with the remote interlock.

7. The system of claim 6, wherein the control device prevents the emission of the EM radiation beam in the absence of the wireless transmit signal and allows the emission of the EM radiation beam when it receives the wireless transmit signal.

8. The system of claim 6, further comprising the EM radiation source including one of a laser or an intense pulse light (IPL) source.

9. The system of claim 8, wherein the laser is selected from the group of lasers consistent of: a CO₂ laser, a Nd-YAG laser, an erbium-based laser, a diode laser, a dye laser, an alexandrite laser, a ruby laser, an argon laser, a class 3B laser, a class 4 laser, a pulsed laser, and a continuous-wave laser.

10. The system of claim 6, further comprising:
an informational signal unit configured to provide at least one of a visual and audible informational signal relating to an operational state of the system.

11. The system of claim 6, further comprising:
an interrogation signal source operable to emit an interrogation signal to the sensor unit; and
wherein the sensor unit is configured to provide the wireless transmit signal as a radio-frequency identification (RFID) signal in response to the interrogation signal.

12. The system of claim 6, wherein the sensor unit includes at least one activation element configured to cause the sensor unit to transmit the wireless transmit signal when the protective eyewear is properly worn by the participant.

13. The system of claim 6, further comprising for multiple participants:
multiple pairs of protective eyewear, one for each participant, the multiple pairs of protective eyewear configured to transmit multiple wireless transmit signals, one from each of the sensor units; and
wherein the control device is configured to allow emission of the EM radiation beam from the EM radiation source when the control device receives the multiple wireless transmit signals, one from each of the multiple pairs of protective eyewear.

14. The system of claim 13, further comprising a detector unit operably connected to the control device and configured to determine a number of the multiple participants.

15. The system of claim 13, wherein the sensor units are configured to provide the wireless transmit signals with different signal features in order to identify different pairs of protective eyewear.

16. An eye protection system for protecting the eyes of at least one participant in an EM radiation-based procedure that employs an EM radiation source, comprising:
the EM radiation source, the EM radiation source having a remote interlock connector and that is configured to emit an EM radiation beam suitable for use in the EM radiation-based procedure;
a control device configured to matingly engage the remote interlock connector and configured to control the emission of the EM radiation beam via the remote interlock connector;
at least one pair of protective eyewear for use by the at least one participant, the at least one pair of protective eyewear configured to at least substantially block the EM radiation from the EM radiation source from reaching the participant's eyes, the at least one pair of protective eyewear having a sensor unit configured to provide a wireless transmit signal to the control device when the at least one protective eyewear is properly worn by the at least one participant; and
wherein the control device enables the remote interlock connect to allow the EM radiation beam to be emitted by the EM radiation source when the control device receives the wireless transmit signal.

17-21. (canceled)

22. The eye protection system of claim 16, wherein the sensor unit further comprises:
a transmitter electronics chip configured to generate the wireless transmit signal;
a power source electrically connected to the transmitter electronics chip to provide power to the transmitter electronics chip; and
an antenna electrically connected to the transmitter electronics chip and configured to communicate to the control device the wireless transmit signal as a wireless transmit signal.

23. The eye protection system of claim 16, further comprising:
an interrogation signal source that emits a radio-frequency identification (RFID) interrogation signal to be received by the sensor unit; and
the sensor unit having an RFID tag that provides the wireless transmit signal in response to the interrogation signal when the eyewear is properly worn.

24. The eye protection system of claim 23, further comprising at least one activation element operably connected to the sensor unit and configured to prevent the RFID tag from providing the transmit signal when the at least one pair of protective eyewear is not properly being worn by the at least one participant.

25. The eye protection system of claim 16, further comprising at least one activation element operably connected to the sensor unit and configured to cause the sensor unit to generate the wireless transmit signal when the protective eyewear is properly worn.

26. (canceled)

27. The eye protection system of claim 25, wherein the at least one activation unit is based on one or more of the following activating properties:
temperature, pressure, light, movement, heartbeat, electrical conductance, height above a floor, and proximity to the EM radiation source.

28. The eye protection system of claim 16, further comprising multiple participants and multiple pairs of protective eyewear, wherein the control device enables the remote interlock connector to allow the EM radiation source to emit the EM radiation beam only when the control device receives wireless transmit signals from each pair of the multiple pairs of protective eyewear respectively worn by the multiple participants.

29. The eye protection system of claim 16, wherein the EM radiation source includes either a laser or an intense pulse light (IPL) source.

30. The eye protection system of claim 29, wherein the laser is selected from the group of lasers comprising: a CO₂ laser, a Nd-YAG laser, an erbium-based laser, a diode laser, a dye laser, an alexandrite laser, a ruby laser, an argon laser, a class 3B laser, a class 4 laser, a pulsed laser, and a continuous-wave laser.

31. (canceled)

32. (canceled)

33. The eye protection system of claim 16, wherein the EM radiation-based procedure includes a medical procedure.

34. (canceled)

35. (canceled)

36. The eye-protection system of claim 16, further comprising a detector unit operably connected to the control device and configured to detect a number of the one or more participants.

37. The eye-protection system of claim 16, further comprising an informational signal unit that generates at least one of a visual and audible informational signal.

38. The eye-protection system of claim 16, wherein the sensor unit is either integrated into or attached onto the at least one pair of protective eyewear.

39. A method of eye protection for a participant in an EM radiation-based procedure that uses an EM radiation source having a remote interlock connector, the EM radiation source being capable of emitting an EM radiation beam, comprising:
operably connecting a control device to the remote interlock connector, the control device being configured to control the emission of the EM radiation beam in response to a wireless transmit signal by enabling or disabling the remote interlock connector; and
transmitting the wireless transmit signal from a sensor unit to the control device, the sensor unit being operably disposed relative to a pair of protective eyewear, with the sensor unit transmitting the wireless transmit signal only when the protective eyewear is properly worn by the participant.

40. The method of claim 39, further comprising:
the control device allowing the emission of the EM radiation beam by enabling the remote interlock connector in response to a transmit signal and preventing the emission of the EM radiation beam in the absence of the transmit signal by disabling the remote interlock connector.

41. (canceled)

42. The method of claim 39, wherein the control device includes first and second operably connected modules, and wherein the method further comprises:
matingly engaging the first module of the control device with the remote interlock connector, the first module configured to perform said control the emission of the EM radiation beam via the remote interlock connector in response to the wireless transmit signal, an electrical transmit signal or an electrical control signal;
receiving the wireless transmit signal from the protective eyewear at the second module and in response generating either the electrical control signal or the electrical transmit signal; and
provide the first module with either the electrical control signal or the electrical transmit signal.

43. The method of claim 39, further comprising:
providing multiple participants each with one of multiple pairs of protective eyewear;
transmitting multiple wireless transmit signals, one from each of the sensor units of the multiple pairs of protective eyewear; and
allowing emission of the EM radiation beam by enabling the remote interlock connector via the control device only when the control device receives wireless transmit signals, one from each of the multiple pairs of protective eyewear.

44. The method of claim 43, further comprising providing the wireless transmit signals from different pairs of protective eyewear with different signal features that identify the different pairs of protective eyewear.

45. The method of claim 43, further comprising comparing a number of the multiple participants to a number of transmit signals received by the control device and not enabling the remote interlock connector unless the number participants is the same as the number of wireless transmit signals.

46. The method of claim 45, further comprising automatically counting the number of multiple participants using a detector unit operably connected to the control device.

47. The method of claim 45, further comprising manually counting the number of multiple participants and providing the manually counted number to the control device.

48. The method of claim 39, further comprising:
providing a visual or audible informational signal representative of an operation state of at least of the control device, the EM radiation source and the protective eyewear.

49. (canceled)

50. The method of claim 39, further comprising providing the wireless transmit signal as a radio-frequency identification (RFID) signal in response to providing an interrogation signal to the sensor unit.

51. (canceled)

52. The method of claim 39, further comprising:
causing the sensor unit to provide the wireless transmit signal in response to an activation element being activated when the protective eyewear is properly worn the participant.

53. The method of claim 39, further comprising carrying out the EM radiation-based procedure as a medical procedure.
